(19) United States
(12) Patent Application Publication
(10) Pub. No.: US 2026/0360481 A1
(43) Pub. Date: Nov. 12, 2026

(54) DECISION TRANSFORMERS FOR GUIDING MATERIAL DISCOVERY

(71) Applicant: International Business Machines Corporation, Armonk, NY (US)

(72) Inventors: Lan Ngoc HOANG, Warrington (GB); CLYDE FARE, Manchester (GB); Lamogha Chiazor, Eastleigh (GB)

(21) Appl. No.: 19/200,602

(22) Filed: May 6, 2025

(51) Int. Cl. G16C 20/50; G06N 3/045; G06N 3/092; G16C 20/70

(52) U.S. Cl. CPC G16C 20/50; G06N 3/045; G06N 3/092; G16C 20/70

(57) ABSTRACT

A plurality of molecules of a dataset are processed using a plurality of ministates, each ministate corresponding to one of the plurality of molecules, the processing based on one or more constraints of one or more given target properties. A next action to perform to generate a modified molecule is selected from a current molecule of the plurality of molecules based on one or more representative rewards and the one or more given target properties. The processing and selecting operations are repeated over M steps to generate a new molecule

### BACKGROUND

The present invention relates generally to the electrical, electronic and computer arts and, more particularly, to machine learning.

Conventional material discovery has been modeled as an optimization or iterative search problem to a direct target objective. Current material discovery uses offline and online reinforcement learning or optimization techniques to guide the search for a molecule that satisfies a target property. A challenge, however, is still how to arrive at a molecule that satisfies the target property, especially when it contains multiple dimensions/objectives.

### BRIEF SUMMARY

Principles of the invention provide systems and techniques for decision transformers for guiding material discovery. In one aspect, an exemplary method includes the operations of processing, using a modified decision transformer, a plurality of molecules of a dataset using a plurality of ministates, each ministate corresponding to one of the plurality of molecules, the processing based on one or more constraints of one or more given target properties; selecting, using the modified decision transformer, a next action to perform to generate a modified molecule from a current molecule of the plurality of molecules based on one or more representative rewards and the one or more given target properties; and repeating the processing and selecting operations over M steps to generate a new molecule.

In one aspect, a computer program product comprises one or more tangible computer-readable storage media and program instructions stored on at least one of the one or more tangible computer-readable storage media, the program instructions executable by a processor, the program instructions comprising processing, using a modified decision transformer, a plurality of molecules of a dataset using a plurality of ministates, each ministate corresponding to one of the plurality of molecules, the processing based on one or more constraints of one or more given target properties; selecting, using the modified decision transformer, a next action to perform to generate a modified molecule from a current molecule of the plurality of molecules based on one or more representative rewards and the one or more given target properties; and repeating the processing and selecting operations over M steps to generate a new molecule.

In one aspect, an apparatus comprises a memory and at least one processor, coupled to the memory, and operative to perform operations comprising processing, using a modified decision transformer, a plurality of molecules of a dataset using a plurality of ministates, each ministate corresponding to one of the plurality of molecules, the processing based on one or more constraints of one or more given target properties; selecting, using the modified decision transformer, a next action to perform to generate a modified molecule from a current molecule of the plurality of molecules based on one or more representative rewards and the one or more given target properties; and repeating the processing and selecting operations over M steps to generate a new molecule.

As used herein, “facilitating” an action includes performing the action, making the action easier, helping to carry the action out, or causing the action to be performed. Thus, by way of example and not limitation, instructions executing on a processor might facilitate an action carried out by instructions executing on a remote processor, chemical process equipment/pharmaceutical manufacturing equipment, or the like, by sending appropriate data or commands to cause or aid the action to be performed. Where an actor facilitates an action by other than performing the action, the action is nevertheless performed by some entity or combination of entities.

Techniques as disclosed herein can provide substantial beneficial technical effects, as will be discussed further below. Features and advantages will become apparent from the following detailed description of illustrative embodiments thereof, which is to be read in connection with the accompanying drawings.

### BRIEF DESCRIPTION OF THE DRAWINGS

The following drawings are presented by way of example only and without limitation, wherein like reference numerals (when used) indicate corresponding elements throughout the several views, and wherein:

FIG. 1 illustrates the generations of a molecule B based on a given molecule A;

FIG. 2 illustrates an offline reinforcement learning environment for designing a molecule B, in accordance with exemplary embodiments;

FIG. 3A shows an abstract depiction of a generic decision transformer with time series of reward/state/action tokens being processed by transformer blocks, in accordance with exemplary embodiments;

FIG. 3B shows a more detailed comparison of the use of a monolithic state vs. a sequence of ministates within a decision transformer, in accordance with exemplary embodiments;

FIG. 4 illustrates a workflow for using a decision transformer to generate a new material, such as a new molecule, in accordance with exemplary embodiments;

FIG. 5 illustrates an exemplary workflow for generating new molecules, in accordance with exemplary embodiments;

FIG. 6 illustrates the generation of the final representative reward(s) and target score, in accordance with exemplary embodiments;

FIG. 7 is a graph of the score vs. the number of training epochs for the decision transformer, in accordance with exemplary embodiments; and

FIG. 8 depicts a computing environment according to an embodiment of the present invention.

It is to be appreciated that elements in the figures are illustrated for simplicity and clarity. Common but well-understood elements that may be useful or necessary in a commercially feasible embodiment may not be shown in order to facilitate a less hindered view of the illustrated embodiments.

### DETAILED DESCRIPTION

Principles of inventions described herein will be in the context of illustrative embodiments. Moreover, it will become apparent to those skilled in the art given the teachings herein that numerous modifications can be made to the embodiments shown that are within the scope of the claims. That is, no limitations with respect to the embodiments shown and described herein are intended or should be inferred.

Given the discussion herein (reference characters refer to the drawings discussed below), in aspects of the invention a method comprises processing, using a modified decision transformer 258, a plurality of molecules 220 of a dataset 304 using a plurality of ministates, each ministate corresponding to one of the plurality of molecules 220, the processing based on one or more constraints of one or more given target properties 312, 316; selecting, using the modified decision transformer 258, a next action to perform to generate a modified molecule 224 from a current molecule 224 of the plurality of molecules 220 based on one or more representative rewards 328 and the one or more given target properties 312, 316 (operation 324); and repeating the processing and selecting operations over M steps to generate a new molecule 224. Technical benefits include a decision transformer (DT) 258 that selects a molecule 220 from a dataset of molecules 220 that satisfies given target molecular properties and constraints in rewards and costs; a DT 258 that uses a target score on a dataset 304 (and trajectories) to train a policy and a corresponding model to construct and/or select a molecule 224 that satisfies the target scores with the underlying dynamics, leveraging known points in the dataset 304 to more effectively inform exploration; smaller embedding matrices for training the decision transformer 258 to generate target molecules 224 resulting in lower memory requirements and faster inference speeds; support for molecular symmetry in molecule selection and generation resulting in an increase in sampling efficiency; multiple rewards implemented using, for example, a Lagrange multiplier, reinforcement learning, multiple agents and other techniques when there are multiple objectives for molecular construction (this can be combined with multi-agents (agentic framework) to coordinate multiple rewards signals instead of a central agent); the use of ministates to provide faster convergence and representations of molecules.

In exemplary embodiments, the one or more representative rewards 328 are generated based on the one or more given target properties 312, 316. Technical benefits include the generation of molecules 224 that satisfy given target molecular properties.

In exemplary embodiments, the generating of the one or more representative rewards 328 is based on the dataset 304. Technical benefits include the generation of rewards based on a training dataset of molecules 220.

In exemplary embodiments, the one or more representative rewards 328 represent the one or more target properties 312, 316 and are combined using a configuration of reward multi-agents. Technical benefits include an improved technique for developing the representative rewards 328.

In exemplary embodiments, the one or more representative rewards 328 represent the one or more target properties 312, 316 and are combined using a Lagrangian multiplier. Technical benefits include an improved technique for developing the representative rewards 328.

In exemplary embodiments, the plurality of ministates is generated from the dataset 304, where each ministate is generated by projecting an original vector of a corresponding molecule 220, 224 to a smaller size vector using a linear transform with learned parameters. The technical benefits include a trained matrix that is typically smaller than the trained matrix that generates a monolithic combined state and vectors that preserve more information than the single vectors of the monolithic combined state.

In exemplary embodiments, the modified decision transformer 258 is updated based on the one or more representative rewards 328 to improve the repeated selecting step (operation 320). Technical benefits include improving the policy of the decision transformer 258.

In exemplary embodiments, the dataset 304 is generated by sequentially selectively modifying one or more of an atom and a bond to an earlier version of a given molecule 220, 224 using a combinatorial set of actions. Technical benefits include adding or removing bonds and/or atoms to a molecule 220.

In exemplary embodiments, the processing, using the modified decision transformer 258, of the plurality of molecules 220 of the dataset 304 further comprises considering molecules 220, 224 on both sides of a given ministate within a block of ministates and considering only earlier molecules 220, 224 when processing molecules 220, 224 from across ministate blocks. The technical benefits include support for molecular symmetry, which requires that the order that the molecules are processed in to generate the state vector should not matter to the decision transformer 258.

In exemplary embodiments, an original dataset is processed into multi-rewards to generate the dataset 304. Technical benefits include the ability to readily generate the dataset 304.

In exemplary embodiments, a related weight of coefficients is changed to weight reward objectives differently. The technical benefits include enabling optimization based on a variety of objectives (depending on the domain).

In exemplary embodiments, a digital representation of the new molecule is sent to a pharmaceutical manufacturer to facilitate synthesis of the new molecule 220, 224. Technical benefits include the manufacture of pharmaceuticals based on the molecule 220, 224.

In one aspect of the invention, a computer program product comprises one or more tangible computer-readable storage media and program instructions stored on at least one of the one or more tangible computer-readable storage media, the program instructions executable by a processor, the program instructions comprising processing, using a modified decision transformer 258, a plurality of molecules 220 of a dataset 304 using a plurality of ministates, each ministate corresponding to one of the plurality of molecules 220, the processing based on one or more constraints of one or more given target properties 312, 316; selecting, using the modified decision transformer 258, a next action to perform to generate a modified molecule 224 from a current molecule 224 of the plurality of molecules 220 based on one or more representative rewards 328 and the one or more given target properties 312, 316 (operation 324); and repeating the processing and selecting operations over M steps to generate a new molecule 224.

In one aspect of the invention, a system comprises a memory and at least one processor, coupled to the memory, and operative to perform operations comprising processing, using a modified decision transformer 258, a plurality of molecules 220 of a dataset 304 using a plurality of ministates, each ministate corresponding to one of the plurality of molecules 220, the processing based on one or more constraints of one or more given target properties 312, 316; selecting, using the modified decision transformer 258, a next action to perform to generate a modified molecule 224 from a current molecule 224 of the plurality of molecules 220 based on one or more representative rewards 328 and the one or more given target properties 312, 316 (operation 324); and repeating the processing and selecting operations over M steps to generate a new molecule 224.

Techniques as disclosed herein can provide substantial beneficial technical effects. Some embodiments may not have these potential advantages and these potential advantages are not necessarily required of all embodiments. By way of example only and without limitation, one or more embodiments may provide one or more of:
a decision transformer (DT) that selects a molecule from a dataset of molecules that satisfies given target molecular properties and constraints in rewards and costs;
a DT that uses a target score on a dataset (and trajectories) to train a policy and a corresponding model to construct and/or select a molecule that satisfies the target scores with the underlying dynamics, leveraging known points in the dataset to more effectively inform exploration;
smaller embedding matrices for training a decision transformer to generate target molecules resulting in lower memory requirements and faster inference speeds;
support for molecular symmetry in molecule selection and generation resulting in an increase in sampling efficiency; and
multiple rewards implemented using, for example, a Lagrange multiplier, reinforcement learning, multiple agents and other techniques when there are multiple objectives for molecular construction (this can be combined with multi-agents (agentic framework) to coordinate multiple rewards signals instead of a central agent).

A decision transformer (DT) is an important machine learning tool for sequential decision making. In exemplary embodiments, a decision transformer architecture is configured to provide guidance towards constructing and/or selecting a molecule that satisfies target molecular properties and constraints. Exemplary techniques combine the formulation of material discovery as a sequential search and define a modified multi-objective decision transformer which provides a search, such as an offline search, based on multiple target objectives. In exemplary embodiments, the decision transformer architecture is performed in three steps:
the decision transformer is given the target molecular properties and constraints in scores and costs;
the DT uses the target score on the offline dataset (and trajectories) to train a policy to reach the target scores with the underlying dynamics; and
implementation of multiple rewards when there are multiple objectives are implemented using, for example, a Lagrange multiplier, reinforcement learning, multiple agents and other techniques.

The output includes a DT that provides a policy for material discovery.

### Material Discovery As an Optimization and Reinforcement Learning Problem

Different approaches have been used to formulate material discovery for better molecular search. Conventional models, especially those using the transformer architecture, have demonstrated good potential in language processing and other modal representations. Conventional techniques include:
optimization: (multi-)objective task to build a molecule with given target properties; and
reinforcement learning: an iterative process to build a molecule from a core/molecule scaffold or to select a molecule candidate from a portfolio (diffusion models are another class of technique for the generative modeling of molecules).

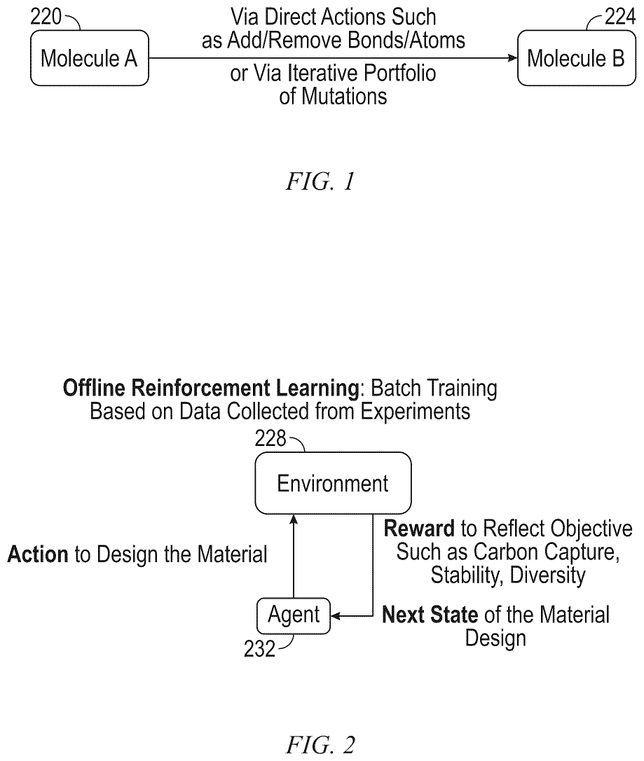

FIG. 1 illustrates the generations of a molecule B 224 based on a given molecule A 220.

The generation may be based on direct actions, such as the addition and/or removal of bonds and/or atoms, or based on an iterative portfolio of mutations. In exemplary embodiments, a decision transformer selects a trajectory (sequence) of actions to generate a molecule B 224 that satisfies a target property(ies).

### Material Discovery as a Markov Decision Process

FIG. 2 illustrates an offline reinforcement learning environment 228 for designing a molecule B 224. In exemplary embodiments, batch training is performed using data collected from experiments. The offline reinforcement learning environment 228 and the online (deployed) learning environment provide (i) a reward to reflect objectives (such as carbon capture, stability, diversity and the like), and (ii) the next state of the material design to an agent 232. The agent 232 then specifies an action, such as add and/or remove a bond and/or an atom, to design the material (such as molecule B 224). With a decision transformer, the material discovery under offline reinforcement learning can be leveraged to turn the search problem into a sequence of actions leading to target rewards that represent multiple target properties.

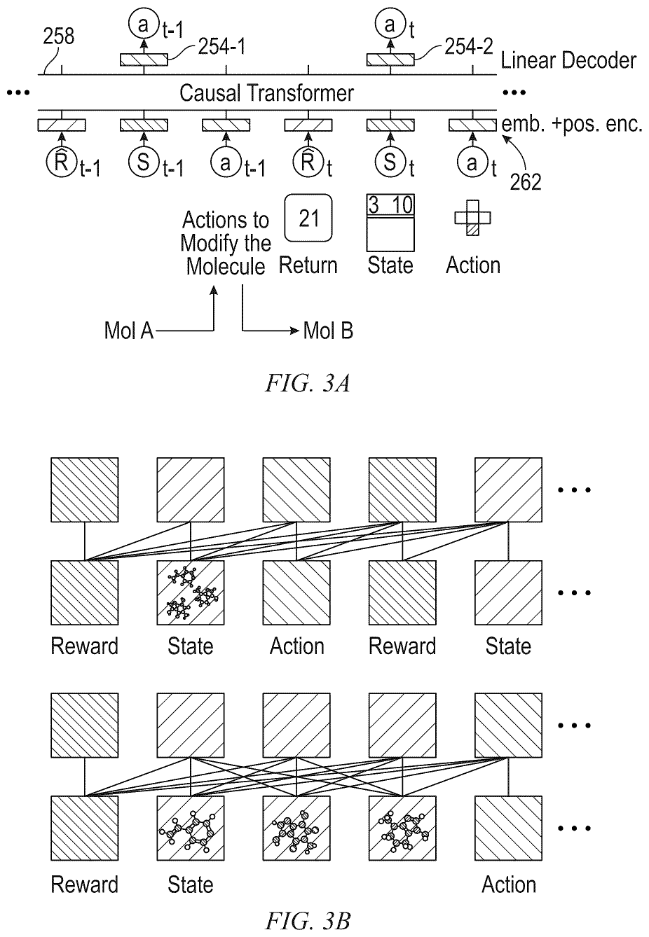

FIG. 3A shows an abstract depiction of a generic decision transformer 258 with time-series of reward/state/action tokens being processed by transformer blocks, in accordance with exemplary embodiments. In the depiction of FIG. 3A, “R” represents reward, “S” represents state, and “a” represents action, each of which is operated on by the embedding and positional encoders 262. The output of the transformer 258 is operated on by the linear decoders 254-1, 254-2.

FIG. 3B shows a more detailed comparison of the use of a monolithic state vs. a sequence of ministates within a decision transformer 258, in accordance with exemplary embodiments. FIG. 3B depicts the differences between monolithic state-blocks (with standard left to right masked attention) and exemplary local ministates (with all-to-all attention within the mini states, except with left to right masked attention between the reward and action tokens and the ministates), in accordance with exemplary embodiments. In FIG. 3B, the top two rows represent the standard decision transformer with the monolithic state. The lower row represents the initial tokens being fed into the transformer while the upper row represents the tokens after an update using the attention block. While only 5 tokens and only one attention-based update are shown in FIG. 3B to avoid clutter, in most cases the tokens would continue on the right and there would typically be many rows above. The lines show which of the input tokens (on the bottom row) are contributing to the output tokens (on the top row). In standard masked attention, only those tokens to the left of a given token contribute to the update. Thus, the first (upper row) token on the left only has contributions from the first (lower row) token; in this case, it is the reward token. While the update to the second token (which, in this case, is the monolithic state) has contributions from both the reward token and the state token. For purposes of clarity, this particular update is shown with bolder lines; that is, how the (monolithic) state token is updated is highlighted.

The bottom two rows of FIG. 3B represent the modified decision transformer 258 where the monolithic state is broken up into separate sub-states (ministates). For illustrative purposes, just three molecules within the state are presented to avoid clutter, but there are typically many more. The update for the second of these two sub-states is highlighted (that is, the second molecule) with bold lines showing the contributions to the updated token while, as before, attention contributions to the other tokens are shown with light lines. Here, all crosshatch-filled ministates are connected to one another. As a result of the operation between connected states, permuting the order of the sub-states consequently makes no difference with respect to generation of the output token.

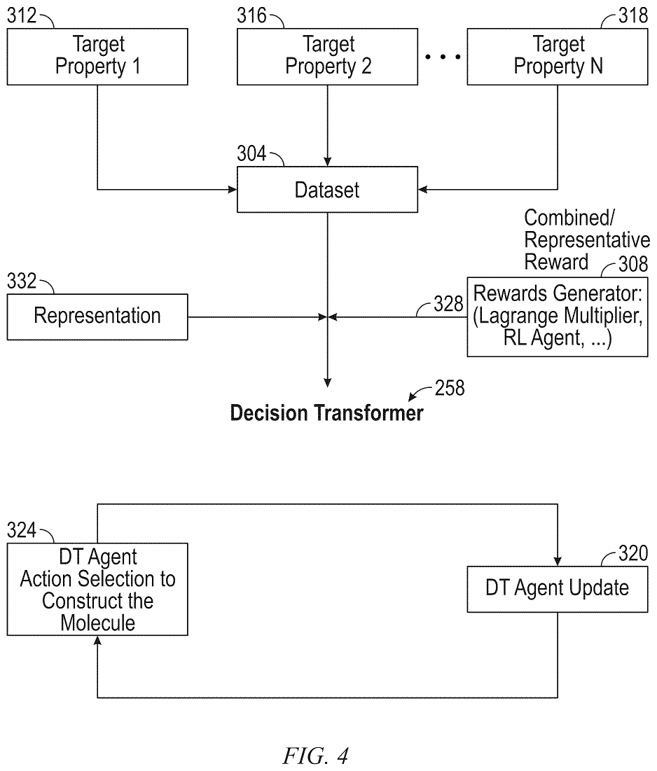

FIG. 4 illustrates a workflow for using a decision transformer 258 (also referred to as a DT agent herein) to generate a new material, such as a new molecule, in accordance with exemplary embodiments. In exemplary embodiments, the decision transformer 258 processes molecules 220 of a dataset 304, such as an offline dataset, making use of a given target property(ies) 312, 316, . . . , 318 and a choice of representation 332. The dataset 304 is also considered in generating a representative reward(s) or combined representative rewards 328 by a rewards generator 308. In exemplary embodiments, the rewards generator 308 utilizes a Lagrange multiplier, a reinforcement learning technique, multiple agent(s) (where each agent may, for example, handle one reward type), or other techniques to generate the combined or representative reward(s) 328. Given the teachings herein, the skilled artisan who would be familiar with machine learning (including reinforcement learning) can select an appropriate mechanism for the rewards generator 308 based on the particular domain of interest.

It is noted that the dataset 304 can be built in a number of ways. In exemplary embodiments, the dataset 304 is built using a combinatorial set of actions, sequentially adding atoms and/or bonds to an earlier version of a molecule using, for example, another reinforcement learning method (such as a Deep-Q Network—DQN). (It is noted that other techniques may be used to construct the sequences present in dataset 304, such as optimization. Generic datasets used for property prediction can also be used, although datasets that do not naturally represent trajectories may result in poorer performance.) In exemplary embodiments, the dataset 304 has a finite size; all of the molecules in the dataset 304 are generated to create a monolithic combined state, which can be very large. For example, a dataset 304 of 1,000 molecules may be generated, where each molecule is represented by a vector of 2,048 bits.

The dataset 304 includes trajectories of molecules and their respective target properties. A means to turn these properties into rewards and some representation 332 for the molecules is selected. In exemplary embodiments, 2,048-bit fingerprints are used (other techniques are contemplated for the above means and for generating the rewards from the target properties); that is, there is separation between the dataset 304 (molecules and properties) and the options for generating the reward and how to represent the molecules. Once the techniques are selected, the data can be processed into a form that can be used to train the decision transformer 258. (It is noted that, in exemplary embodiments, the dataset 304 is used to train the decision transformer 258 and thus the dataset 304 does not increase in size. However, once the decision transformer 258 is trained, it can be used in a ‘live’ setting in which the decision transformer 258 will suggest new molecules that do not exist in the dataset 304 (if there are means to calculate or generate the properties of these molecules via simulation or experiment). The dataset 304 can then be enhanced using these results.

Conventionally, the state representations within dataset 304 which are characterized by multiple molecules are shrunk down to a single monolithic smaller sized vector per state (state vector). For example, the states within dataset 304 can be projected to a 128-bit vector using a trained matrix (linear transform with learned parameters), but with a corresponding loss in information. Moreover, it is noted that it is desirable that molecular symmetry be supported, which requires that the order that the molecules are processed in to generate the state vector should not matter to the DT 258; however, the monolithic combined state described above, however, does not support such molecular symmetry as the order of the molecules impacts the resulting representation. (Since the monolithic state is generated by concatenating and then simplifying the representations for multiple molecules, the order that the molecules are present within the state necessarily impacts the final monolithic representation (that is, switching the order of two of the molecules will alter the final monolithic state representation). This violates the permutation invariance symmetry that is expected (since the agent is being asked to pick a molecule out of multiple molecules, the answer should not depend on the order with which the options are presented.))

In exemplary embodiments, the dataset 304 is used to generate a set of ministates, where each ministate corresponds to one of the molecules of the dataset 304. Each ministate is generated by projecting the original vector of a given molecule 220, 224 to a smaller vector size, such as from 2,048 bits to 128 bits using a trained matrix (linear transform with learned parameters). The trained matrix is typically smaller than the trained matrix that generates the monolithic combined state. The result is 1,000 128-bit vectors which preserve more information than the single 128-bit vector of the monolithic combined state.

Once generated, rather than performing sequences of reward, monolithic state, action, reward, monolithic state, action, and so on using DT 258, instead, sequences of reward, ministate1, ministate2, . . . ministateN, action, reward, ministate1, ministate, . . . , ministateN and so on are performed using the DT 258. When generating molecule 224, DT 258 makes use of an explicit representation of all 1,000 ministates; this contains more information than molecules 224 generated using the monolithic combined state.

Moreover, the self-attention mechanism of the DT 258 is modified to support molecular symmetry. Conventionally, only tokens on the left (earlier molecules) are considered by the self-attention mechanism when considering a given token, thus not preserving molecular symmetry. In exemplary embodiments, the ministates corresponding to the molecules on both sides of a given molecule (within a block of ministates) are considered (that is, there is no notion of order), while only ministates corresponding to the molecules on the left (earlier tokens) are considered when dealing with molecules that reside across blocks of ministates (see, FIG. 3A).

In exemplary embodiments, a DT agent 258 selects a next action to perform to construct the molecule 224 based on the combined or representative reward(s) 328 and the target property(ies) 312, 316 (operation 324). The combined, or representative, reward(s) 328 are also used to update the DT agent 258 to improve the selection process (operation 320).

In exemplary embodiments, given a reward(s) and target property(ies) 312, 316, the DT 258 performs M steps to generate a molecule 224. Each step processes all of the ministates generated from the dataset 304 (such as 1,000 ministates) and each step results in the definition of a single action in selecting and/or constructing a modified molecule 220, 224. Once the M steps are performed (also referred to as a trajectory of M=50 steps) and the M corresponding actions are implemented, a single molecule 224 is generated.

### Representation of Material Molecules

In exemplary embodiments, a material molecule 220, 224 is represented using a particular format, such as a three-dimensional (3D) coordinates or string format (as is common in conventional molecular action spaces) in the context of generating new molecules. (It is noted that there are two representations utilized in the above techniques. First is the representation in the context of generating the state (either monolithic or mini), which is some set of numbers that the decision transformer 258 can use to determine the action to perform (such as what new molecule to propose). These are numerical representations, that is, vectors of numbers. Second is the representation in the context of the way the decision transformer 258 can take actions, such as how it can propose new molecules. In exemplary embodiments, string representations of a molecule are used; that is, the decision transformer 258 can propose a new molecule by adding a new character to a growing string. In exemplary embodiments, a space of molecules to explore that does not use an explicit representation such as the string representations can be defined. The decision transformer 258 is compatible with numerous viable combinations of such techniques.)

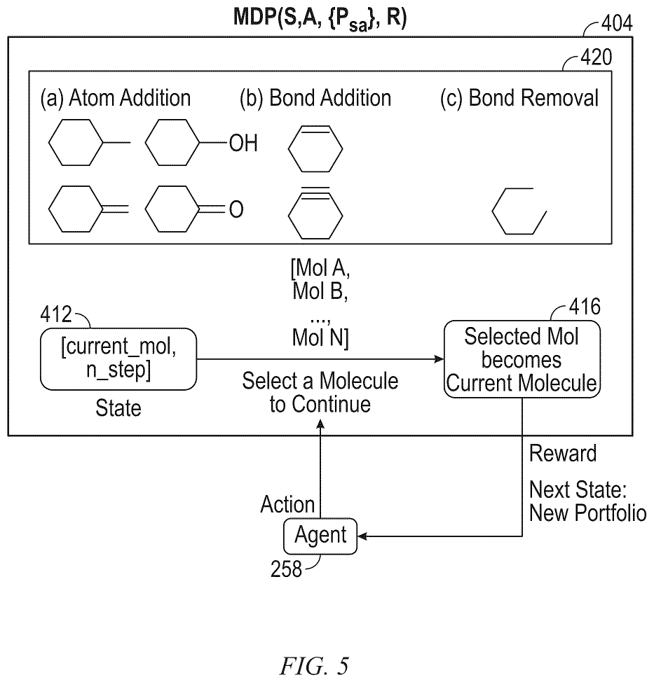

FIG. 5 illustrates an exemplary workflow 404 for generating new molecules 224, in accordance with exemplary embodiments. Each molecule 224 is represented by a given structure 420. A current molecule 224 at a current step (n_step) represents a current state 412 that is used by the DT agent 258 to select a new molecule 224 from the dataset 304 based on a given reward(s) (operation 416). The selected molecule 224 becomes the current molecule 224 of the next step.

### Different Target Properties for Material Discovery Objectives

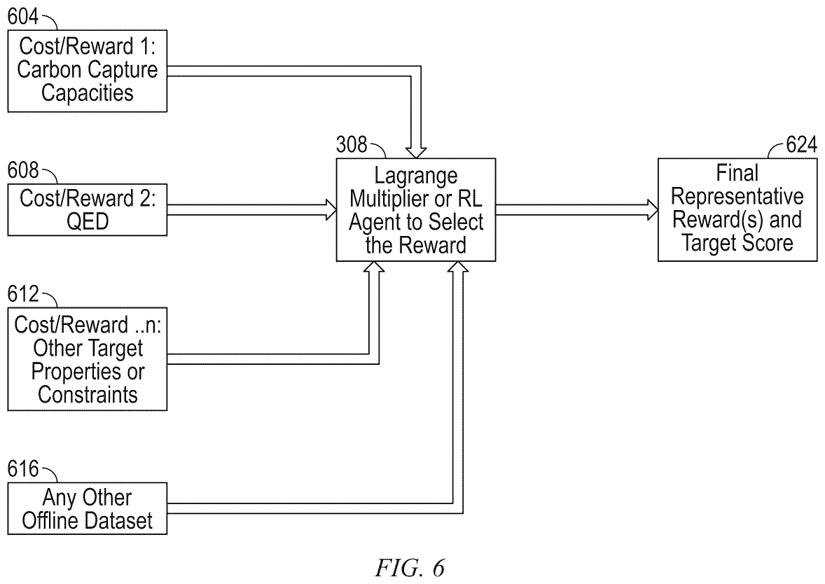

FIG. 6 illustrates the generation of the final representative reward(s) and target score 624, in accordance with exemplary embodiments. As described above, the rewards generator 308 utilizes a Lagrange multiplier, reinforcement learning, multiple agents, or other technique to generate the final representative reward(s) and target score 624. The arbitrary target score of the DT 258 is replaced with representative material properties. Combinations of rewards and different levels of fidelity (different rewards accuracy) and uncertainties are also allowed. Exemplary costs/rewards include carbon capture capacities 604, quantitative estimate of drug-likeness (QED) 608, other target properties or constraints 612, and/or any other offline dataset 616. (In principle, in addition to choosing a new molecule, an agent could choose a new molecule and a particular choice of experimental modality with which to evaluate the molecule. This can allow an agent to choose to make use of less expensive although less accurate measures of the target properties.)

In some instances, an example of material discovery for a target QED using a conventional representation (in the context of molecules) is implemented on a discovery pipeline.

### Sample Results

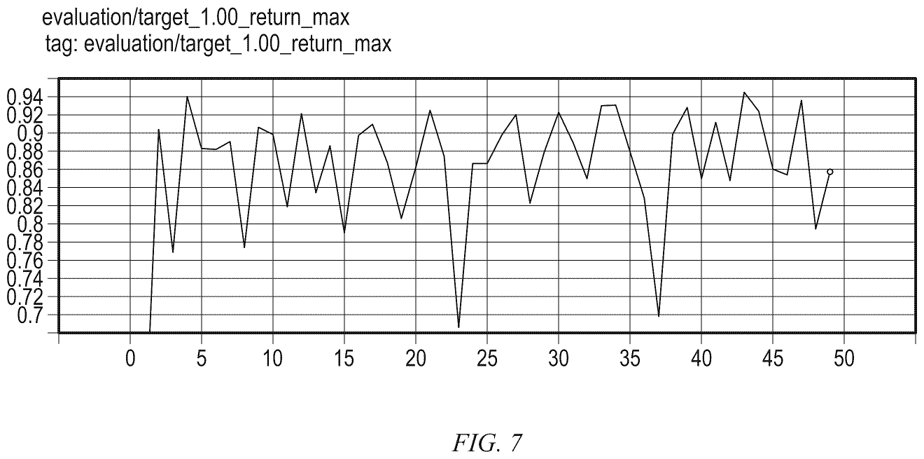

FIG. 7 is a graph of the score vs. the number of training epochs for the decision transformer 258, in accordance with exemplary embodiments. The decision transformer 258 results show that the DT 258 is capable of reaching ~0.94 for the final step reward in four epochs of training (0.944 was also reached at epoch 43). The graph was generated using a full 35,000-episode dataset 304 generated by the deep Q-network (DQN) agent using the molecular environment (see FIG. 5 and the associated text) applied to the QED reward (the maximum data point in that dataset is 0.948). As illustrated in FIG. 7, exemplary embodiments quickly reached the maximum score (within four training epochs).

It is noted that each epoch relates to one complete run through the training dataset 304, and the creation of one molecule 224 following the training. In exemplary embodiments, the training is conducted for a given number of runs, for a given amount of time and the like. The DT 258 having the best results is then selected for deployment. In exemplary embodiments, training continues until the results become stable (the fluctuations of FIG. 7 shrink or stay within a range of a moving average window; the skilled artisan can select a threshold heuristically depending on the domain). In any case, the DT 258 can be retrained as needed. For example, the DT 258 can be retrained when the dataset 304 is no longer consistent with the target property(ies).

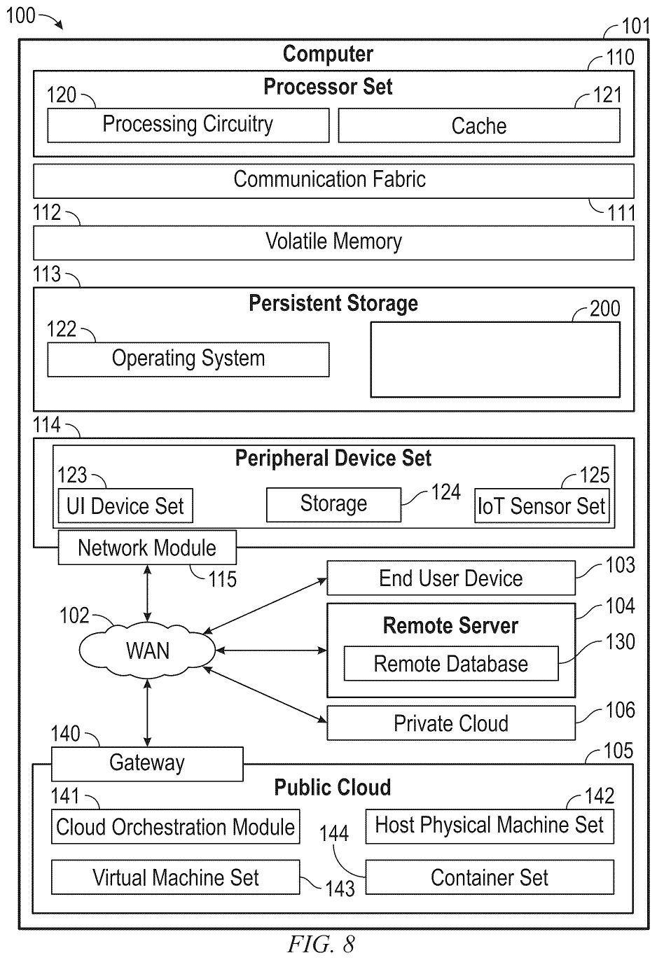

Refer now to FIG. 8.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

Computing environment 100 contains an example of an environment for the execution of at least some of the computer code involved in performing the inventive methods, such as material design machine learning system 200. The code in block 200 can communicate, for example, with chemical process equipment/pharmaceutical manufacturing equipment, or the like, as an end user device, over WAN 102 or the like, to facilitate synthesis of molecules; the molecules are then synthesized by the equipment. In addition to block 200, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and block 200, as identified above), peripheral device set 114 (including user interface (UI) device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 9. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in block 200 in persistent storage 113.

COMMUNICATION FABRIC 111 is the signal conduction path that allows the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, volatile memory 112 is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open source Portable Operating System Interface-type operating systems that employ a kernel. The code included in block 200 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion-type connections (for example, secure digital (SD) card), connections made through local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN 102 may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method comprising:
processing, using a modified decision transformer, a plurality of molecules of a dataset using a plurality of ministates, each ministate corresponding to one of the plurality of molecules, the processing based on one or more constraints of one or more given target properties;
selecting, using the modified decision transformer, a next action to perform to generate a modified molecule from a current molecule of the plurality of molecules based on one or more representative rewards and the one or more given target properties; and
repeating the processing and selecting operations over M steps to generate a new molecule.

2. The computer-implemented method of claim 1, further comprising generating the one or more representative rewards based on the one or more given target properties.

3. The computer-implemented method of claim 2, wherein the generating of the one or more representative rewards is based on the dataset.

4. The computer-implemented method of claim 2, wherein the one or more representative rewards represent the one or more target properties and are combined using a configuration of reward multi-agents.

5. The computer-implemented method of claim 2, wherein the one or more representative rewards represent the one or more target properties and are combined using a Lagrangian multiplier.

6. The computer-implemented method of claim 1, further comprising generating the plurality of ministates from the dataset, where each ministate is generated by projecting an original vector of a corresponding molecule to a smaller size vector using a linear transform with learned parameters.

7. The computer-implemented method of claim 1, further comprising updating the modified decision transformer based on the one or more representative rewards 328 to improve the repeated selecting step.

8. The computer-implemented method of claim 1, further comprising generating the dataset by sequentially selectively modifying one or more of an atom and a bond to an earlier version of a given molecule using a combinatorial set of actions.

9. The computer-implemented method of claim 1, wherein the processing, using the modified decision transformer, of the plurality of molecules of the dataset further comprises considering molecules on both sides of a given ministate within a block of ministates and considering only earlier molecules when processing molecules from across ministate blocks.

10. The computer-implemented method of claim 1, further comprising processing an original dataset into multi-rewards to generate the dataset 304.

11. The computer-implemented method of claim 1, further comprising changing a related weight of coefficients to weight reward objectives differently.

12. The computer-implemented method of claim 1, further comprising sending a digital representation of the new molecule to a pharmaceutical manufacturer to facilitate synthesis of the new molecule.

13. A computer program product, comprising:
one or more tangible computer-readable storage media and program instructions stored on at least one of the one or more tangible computer-readable storage media, the program instructions executable by a processor, the program instructions comprising:
processing, using a modified decision transformer, a plurality of molecules of a dataset using a plurality of ministates, each ministate corresponding to one of the plurality of molecules, the processing based on one or more constraints of one or more given target properties;
selecting, using the modified decision transformer, a next action to perform to generate a modified molecule from a current molecule of the plurality of molecules based on one or more representative rewards and the one or more given target properties; and
repeating the processing and selecting operations over M steps to generate a new molecule.

14. A system comprising:
a memory; and
at least one processor, coupled to said memory, and operative to perform operations comprising:
processing, using a modified decision transformer, a plurality of molecules of a dataset using a plurality of ministates, each ministate corresponding to one of the plurality of molecules, the processing based on one or more constraints of one or more given target properties;
selecting, using the modified decision transformer, a next action to perform to generate a modified molecule from a current molecule of the plurality of molecules based on one or more representative rewards and the one or more given target properties; and
repeating the processing and selecting operations over M steps to generate a new molecule.

15. The system of claim 14, the operations further comprising generating the one or more representative rewards based on the one or more given target properties.

16. The system of claim 15, wherein the one or more representative rewards represent the one or more target properties and are combined using a configuration of reward multi-agents.

17. The system of claim 14, the operations further comprising generating the plurality of ministates from the dataset, where each ministate is generated by projecting an original vector of a corresponding molecule to a smaller size vector using a linear transform with learned parameters.

18. The system of claim 14, the operations further comprising updating the modified decision transformer based on the one or more representative rewards to improve the repeated selecting step.

19. The system of claim 14, the operations further comprising generating the dataset by sequentially selectively modifying one or more of an atom and a bond to an earlier version of a given molecule using a combinatorial set of actions.

20. The system of claim 14, wherein the processing, using the modified decision transformer, of the plurality of molecules of the dataset further comprises considering molecules on both sides of a given ministate within a block of ministates and considering only earlier molecules when processing molecules from across ministate blocks.
